(19) United States
(12) Patent Application Publication
(10) Pub. No.: US 2026/0069782 A1
(43) Pub. Date: Mar. 12, 2026

(54) Multi-Chamber Syringe With Multiple Plungers for Sequential Delivery of Fluids and Methods of Use

(71) Applicant: Becton, Dickinson and Company, Franklin Lakes, NJ (US)

(72) Inventors: Rahul Malviya, Salmon Arm (CA); Aman Desai, Bengaluru (IN); Hemant Vilas Belsare, Pune (IN); Amarsinh Deeliprao Jadhav, Hyderabad (IN)

(21) Appl. No.: 18/868,193

(22) PCT Filed: Jun. 15, 2023
(86) PCT No.: PCT/US2023/025357

§ 371 Date: Nov. 22, 2024

(30) Foreign Application Priority Data
Jun. 24, 2022 (IN) 202211036490

(51) Int. Cl. A61M 5/19; A61M 5/178; A61M 5/315

(52) U.S. Cl. CPC A61M 5/19; A61M 5/31501; A61M 2005/1787; A61M 2005/31508

(57) ABSTRACT

A multi-chamber syringe for sequential expulsion of a first fluid followed by a second fluid includes a container having a proximal end and a distal end including a fluid port for expulsion of the fluids the container. The container defines a first chamber for containing the first fluid and a second chamber for containing the second fluid. The syringe also includes: a first plunger for expelling the first fluid from the first chamber; a second plunger for expelling the second fluid from the second chamber; and a valve positioned in the container. The valve is configured to transition between a first position, where fluid flow from the first chamber through the valve is prevented, and a second position, where fluid flow from the first chamber through the at least one valve to the fluid port for expelling the first fluid from the container is permitted.

### CROSS-REFERENCE TO RELATED APPLICATION

The present application claims priority to Indian Patent Application No. 202211036490 entitled “Multi-Chamber Syringe with Multiple Plungers for Sequential Delivery of Fluids and Methods of Use” filed Jun. 24, 2022, the entire disclosure of which is hereby incorporated by reference in its entirety.

### BACKGROUND OF THE INVENTION

### Field of the Invention

The present disclosure relates generally to syringes for expelling multiple fluids and, in particular, to a syringe for sequential expulsion of a flush solution and a medical fluid.

### Description of Related Art

Vascular access devices (VADs) are commonly used medical devices, which can include intravenous (IV) catheters, such as peripheral catheters or central venous catheters. If not properly maintained or if exposed to a non-sterile environment, the VADs can become contaminated, sealed with blood clots, and/or can spread infection. Many medical facilities implement sterile practices and protocols to ensure that VADs are used properly and do not become sealed or infected. These protocols often include sterilizing the VAD and flushing the catheter with a flush solution. Specifically, VAD standards of practice usually recommend flush procedures be performed after catheter placement, before fluid infusion, and before and after drug administration, blood sampling, transfusions, and/or administration of parenteral nutrition.

These flush procedures are intended to confirm catheter patency, avoid drug incompatibilities, ensure that the complete drug dose is administered to the patient, prevent thrombus formation, and minimize a risk of bloodstream infections caused by contamination of the VAD. Further, flushing can prevent build-up of deposits of blood, blood residue, and IV drugs within a catheter or other VAD device. Such build-up can cause partial or complete blockage of the fluid pathway in a catheter system, which may require a practitioner to perform certain expensive and potentially dangerous processes for purging the affected catheter or may require the practitioner to exchange the blocked catheter with a new catheter. Often, such blockages lead to interruptions in therapy, which may compromise patient care. The build-up of residue within a catheter may also increase infection risk.

During administration of a drug or another medical fluid, a catheter or VAD is usually flushed both before and after delivery of the fluid from a syringe or from another drug delivery device to the VAD. In some cases, catheters can be flushed using syringe assemblies that are pre-filled with various fluids (referred to herein as flush solutions). The size of the pre-filled syringe used to flush the catheter varies based, for example, on a size and length of the catheter. Typically, pre-filled syringes for flushing a catheter or IV line contain a volume of about 1 mL, 3 mL, 5 mL, or 10 mL of flush solution. Many different syringes commercially available from different manufacturers can be used for providing the flush solution through a catheter or IV line of a patient including, for example, the BD PosiFlush™ pre-filled saline syringe by Becton, Dickinson and Company.

In order to perform a flush procedure, a practitioner connects the pre-filled flush syringe to the VAD and advances a plunger rod into a barrel of the pre-filled syringe, which expels the flush solution through a nozzle or fluid port of the pre-filled syringe to the VAD. After flushing, the pre-filled syringe is disconnected from the VAD so that the practitioner can administer a dose of the medical fluid. The medical fluid is conventionally contained in a vial and is drawn into a separate syringe which, after being filled with the medical fluid, is connected to the VAD. The medical fluid is then injected to the VAD by advancing a plunger rod through the syringe containing the medical fluid. After the medical fluid is injected to the VAD, the syringe is disconnected from the VAD and a second pre-filled flush syringe is used to flush the VAD. Flushing the VAD after administering the medical fluid confirms patency of the catheter and advances any remaining medical fluid in the catheter to the patient ensuring that a full dose of the medical fluid is delivered to the patient.

Connecting or disconnecting a syringe or another device from the VAD introduces portions of the VAD to an unsterile outside environment, which increases a possibility that the VAD will become contaminated and/or will be a source of a bloodstream infection. In order to decrease a risk of bloodstream infection and to ensure VADs are used and maintained correctly, standards of practice require disinfecting and cleaning any fluid ports or connectors of the VAD each time that a new syringe is connected to the VAD. Accordingly, reducing a number of syringes that must be connected to the VAD during a drug delivery procedure simplifies drug administration by reducing a number of times that portions of the VAD must be disinfected and cleaned. Reducing the number of syringes or other fluid delivery devices used during a procedure also reduces opportunities for portions of the VAD to become contaminated, thereby reducing the risk for bloodstream infections. The syringes and methods of the present disclosure provide simplified drug administration procedures by reducing the number of syringes used during such procedures, thereby reducing a risk of contamination and bloodstream infection.

### SUMMARY OF THE INVENTION

According to an aspect of the disclosure, a multi-chamber syringe for sequential expulsion of at least a first fluid followed by a second fluid includes a container having a proximal end and a distal end including a fluid port for expulsion of the first fluid and the second fluid from the container. The container defines a first chamber for containing the first fluid and a second chamber for containing the second fluid. The syringe also includes: a first plunger for expelling the first fluid from the first chamber; a second plunger for expelling the second fluid from the second chamber; and at least one valve positioned in the container. The at least one valve is configured to transition between a first position, where fluid flow from the first chamber through the valve is prevented, and a second position, where fluid flow from the first chamber through the at least one valve to the fluid port for expelling the first fluid from the container is permitted.

According to another aspect of the disclosure, a pre-filled flushing syringe includes any of the previously described syringes and a predetermined volume of the first fluid disposed in the first chamber of the pre-filled syringe. The valve is initially in the first position, thereby preventing the first fluid disposed in the first chamber from passing from the first chamber through the at least one valve.

According to another aspect of the disclosure, a method for sequential expulsion of fluids from one of the previously described syringes includes: moving the valve to the second position, such that fluid flow from the first chamber through the valve to the fluid port is permitted; moving the first plunger through the first chamber to expel the first fluid from the first chamber and through the fluid port; moving the at least one valve to the first position, such that fluid flow from the second chamber through the at least one valve to the fluid port is permitted; and moving the second plunger through the second chamber to expel the second fluid from the second chamber and through the fluid port.

According to another aspect of the disclosure, a syringe includes a housing having an upper barrel and a lower barrel. The upper barrel includes an open proximal end, a distal end, a sidewall extending between the proximal end and the distal end, and a partition extending axially through the barrel separating the barrel into a first chamber configured to contain a first fluid and a second chamber configured to contain a second fluid. The lower barrel is connected to the distal end of the upper barrel comprising a fluid port. The syringe also includes a first plunger for expelling the first fluid from the first chamber; a second plunger for expelling the second fluid from the second chamber; and a manually operable valve positioned in the lower barrel. The manually operable valve is configured to be moved between a first position, where fluid flow from the second chamber through the valve to the fluid port for expelling the second fluid from the syringe is permitted, and a second position, where fluid flow from the first chamber through the valve to the fluid port for expelling the first fluid from the syringe is permitted.

According to another aspect of the disclosure, a syringe includes a barrel having an open proximal end, a distal end including a frustoconical portion and a fluid port, a cylindrical sidewall between the proximal end and the distal end, and a partition extending axially through the barrel separating the barrel into a first chamber configured to contain the first fluid and a second chamber configured to contain the second fluid. The syringe also includes a first plunger for expelling the first fluid from the first chamber, a second plunger for expelling the second fluid from the second chamber, and a compressible bellows disposed in the first chamber. The compressible bellows is configured to move between an unbiased position, where fluid flow from the first chamber is prevented, and a biased position, where fluid flow from the first chamber to the fluid port is permitted.

Non-limiting illustrative examples of embodiments of the present disclosure will now be described in the following numbered clauses.

Clause 1: A multi-chamber syringe for sequential expulsion of at least a first fluid followed by a second fluid, the syringe comprising: a container comprising a proximal end and a distal end comprising a fluid port for expulsion of the first fluid and the second fluid from the container, wherein the container defines a first chamber for containing the first fluid and a second chamber for containing the second fluid; a first plunger for expelling the first fluid from the first chamber; a second plunger for expelling the second fluid from the second chamber; and at least one valve positioned in the container configured to transition between a first position, where fluid flow from the first chamber through the at least one valve is prevented, and a second position, where fluid flow from the first chamber through the at least one valve to the fluid port for expelling the first fluid from the container is permitted.

Clause 2: The syringe of clause 1, wherein the first chamber and the second chamber extend axially through the container, with a longitudinal axis of the first chamber being substantially parallel to a longitudinal axis of the second chamber.

Clause 3: The syringe of clause 1 or clause 2, wherein, with the at least one valve in the first position, fluid flow from the second chamber through the at least one valve to the fluid port for expelling the second fluid from the container is permitted, and with the at least one valve in the second position fluid flow from the second chamber through the at least one valve is prevented.

Clause 4: The syringe of any of clauses 1-3, wherein, with the at least one valve in the first position, moving the second plunger proximally through the second chamber aspirates the second fluid into the second chamber.

Clause 5: The syringe of any of clauses 1-4, wherein the container comprises an upper barrel comprising the first chamber and the second chamber, and a lower barrel connected to a distal end of the upper barrel enclosing the at least one valve.

Clause 6: The syringe of clause 5, wherein the upper barrel comprises: an open proximal end, a distal end, an annular sidewall extending between the open proximal end and the open distal end, and an axially extending partition extending from the open proximal end to the distal end of the upper barrel separating the first chamber from the second chamber.

Clause 7: The syringe of clause 5 or clause 6, further comprising at least one seal covering the open distal end of the upper barrel, the at least one seal comprising a first opening for permitting the first fluid to flow from the first chamber to the at least one valve, when the at least one valve is in the second position, and a second opening for permitting the second fluid to flow from the second chamber through the at least one valve, when the at least one valve is in the first position.

Clause 8: The syringe of clause 7, wherein the at least one seal comprises a proximal seal positioned between the distal end of the upper barrel and the at least one valve, and a distal seal positioned between the at least one valve and a distal end of the lower barrel.

Clause 9: The syringe of clause 7 or clause 8, wherein the first plunger and/or the second plunger comprise a stopper having a flat distal surface and a protrusion extending from the flat distal surface positioned to insert into the first opening or the second opening to fully expel the first fluid from the first chamber or the second fluid from the second chamber.

Clause 10: The syringe of any of clauses 5-9, wherein the upper barrel is connected to the lower barrel by ultrasonic welding.

Clause 11: The syringe of any of clauses 1-10, wherein the upper barrel and the lower barrel comprise at least one of polyester, polycarbonate, polypropylene, polyethylene, polyethylene terephthalate, or acrylonitrile butadiene styrene.

Clause 12: The syringe of any of clauses 1-11, wherein the first plunger comprises a first stopper in the first chamber and a first plunger rod connected to and extending from the first stopper through the proximal end of the container.

Clause 13: The syringe of clause 12, wherein the first plunger rod comprises a tab configured to contact a portion of the container as the first rod is being moved through the first chamber, thereby providing feedback that an initial volume of the first fluid has been expelled from the first chamber.

Clause 14: The syringe of clause 12 or clause 13, wherein the second plunger comprises a second stopper in the second chamber and a second plunger rod connected to and extending from the second stopper through the proximal end of the container.

Clause 15: The syringe of clause 14, wherein the first chamber and the second chamber have a semi-circular cross-sectional shape, and wherein the first stopper and the second stopper have a semi-circular flat distal surface corresponding to the cross-sectional shape of the first chamber or the second chamber.

Clause 16: The syringe of clause 14 or clause 15, further comprising at least one removable cap engaged between the container and the first plunger rod and/or the second plunger rod for preventing movement of the plunger rods until the at least one removable plunger cap is removed from the syringe.

Clause 17: The syringe of any of clauses 14-16, wherein the first stopper and/or the second stopper comprise a thermoplastic elastomer, such as isoprene.

Clause 18: The syringe of any of clauses 14-17, wherein the first stopper and/or the second stopper comprise a proximal end, a distal end, an outer surface extending between the proximal end and the distal end that seals against an inner surface of the container.

Clause 19: The syringe of any of clauses 14-18, wherein the first plunger rod and/or the second plunger rod comprise a connector portion inserted into and engaged to a cavity of the first stopper and/or the second stopper for securing the first stopper and/or the second stopper to the first plunger rod and/or the second plunger rod.

Clause 20: The syringe of any of clauses 14-19, wherein the at least one valve comprises a body comprising a channel which is fluidly connected to the second chamber when the at least one valve is in the first position and fluidly connected to the first chamber when the at least one valve is in the second position.

Clause 21: The syringe of clause 20, wherein the body comprises a rotating disc and a knob extending through an opening of the container for rotating the disc between the first position and the second position.

Clause 22: The syringe of clause 21, wherein the disc is rotated between 80 degrees and 120 degrees between the first position and the second position.

Clause 23: The syringe of any of clauses 1-4, wherein the container comprises a barrel comprising an annular sidewall, a frustoconical portion extending from the annular sidewall to the fluid port, and a partition extending through the barrel that separates the first chamber from the second chamber.

Clause 24: The syringe of clause 23, wherein the first plunger comprises a first stopper comprising a substantially flat distal surface and a first plunger rod connected to and extending proximally from the first stopper.

Clause 25: The syringe of clause 24, wherein the second plunger comprises a second stopper comprising a half conical distal surface sized to be received within the frustoconical portion of the barrel and a second plunger rod connected to and extending proximally from the second stopper.

Clause 26: The syringe of clause 25, wherein the first plunger rod comprises a first tab positioned proximate to a distal end of the first plunger rod configured to contact the barrel to prevent the first plunger rod from being moved distally through the first chamber to expel the first fluid from the first chamber.

Clause 27: The syringe of clause 26, wherein the second plunger rod comprises a second tab positioned proximate to a proximal end of the second plunger rod configured to contact and depress the first tab so that the first plunger rod can be moved through the first chamber.

Clause 28: The syringe of any of clauses 23-27, wherein the at least one valve comprises a compressible body which, in an expanded position, prevents fluid flow from the first chamber to the fluid port, and in a compressed position permits fluid flow from the first chamber to the fluid port.

Clause 29: The syringe of clause 28, wherein the barrel comprises at least one slot defining a flow channel for the fluid flow from the first chamber to the fluid port, and wherein the at least one slot is positioned such that when the compressible body is in the expanded position, fluid flow through the at least one slot is prevented and when the compressible body is in the compressed position, fluid flow through the at least one slot to the fluid port is permitted.

Clause 30: The syringe of clause 29, wherein the at least one slot is an opening extending through the partition of the barrel, and wherein, when the second stopper is in a distal-most position in the second chamber, the second stopper seals a portion of the at least one slot preventing the first fluid from flowing into the second chamber.

Clause 31: The syringe of any of clauses 28-30, wherein the compressible body comprises a half conical distal surface configured to be positioned in the frustoconical portion of the barrel and at least one radially extending rib configured to contact an inner surface of the cylindrical sidewall of the barrel to seal the first fluid within the first chamber.

Clause 32: A pre-filled flushing syringe, comprising: the syringe of any of clauses 1-31;and a predetermined volume of the first fluid disposed in the first chamber of the pre-filled syringe, wherein the at least one valve is initially in the first position, thereby preventing the first fluid disposed in the first chamber from passing from the first chamber through the at least one valve.

Clause 33: The pre-filled syringe of clause 32, wherein the first fluid is a saline flush solution.

Clause 34: The pre-filled syringe of clause 32 or clause 33, further comprising a removable plunger cap engaged between the first plunger and/or the second plunger and the container for preventing movement of the plungers until the cap is removed.

Clause 35: A method for sequential expulsion of fluids from the syringe of any of clauses 1-31, the method comprising: moving the at least one valve to the second position, such that fluid flow from the first chamber through the at least one valve to the fluid port is permitted; moving the first plunger through the first chamber to expel the first fluid from the first chamber and through the fluid port; moving the at least one valve to the first position, such that fluid flow from the second chamber through the at least one valve to the fluid port is permitted; and moving the second plunger through the second chamber to expel the second fluid from the second chamber and through the fluid port.

Clause 36: The method of clause 35, further comprising initially, with the at least one valve in the first position, aspirating the second fluid into the second chamber by moving the second plunger distally through the second chamber.

Clause 37: The method of clause 36, wherein aspirating the second fluid further comprises attaching a needle to the fluid port of the container, inserting the needle into a fluid container, and drawing the second fluid from the fluid container to the second chamber of the syringe.

Clause 38: The method of any of clauses 35-37, wherein the syringe is provided partially prefilled containing a predetermined volume of the first fluid in the first chamber.

Clause 39: The method of any of clauses 35-38, further comprising attaching at least one patient line to the fluid port of the syringe prior to expelling the second fluid from the second chamber and through the fluid port.

Clause 40: The method of any of clauses 35-39, wherein the first fluid comprises a flush solution, such as saline solution and/or heparin lock flush solution, and the second fluid comprises a therapeutic agent.

Clause 41: The method of any of clauses 35-40, wherein the first plunger comprises a plunger rod comprising a tab configured to contact a portion of the container as the first plunger rod is being moved through the first chamber, thereby providing feedback that an initial volume of the first fluid has been expelled from the first chamber, and wherein moving the first plunger through the first chamber to expel the first fluid from the first chamber and through the fluid port comprises: moving the first plunger through the first chamber to expel the initial volume of the first fluid until the tab contacts the container prior to expelling the second fluid; and after expelling the second fluid from the second chamber, continuing to move the first plunger through the first chamber to expel a remaining volume of the first fluid from the first chamber.

Clause 42: A syringe comprising: a housing comprising an (i) upper barrel comprising an open proximal end, a distal end, a sidewall extending between the proximal end and the distal end, and a partition extending axially through the barrel separating the barrel into a first chamber configured to contain a first fluid and a second chamber configured to contain a second fluid, and (ii) a lower barrel connected to the distal end of the upper barrel comprising a fluid port; a first plunger for expelling the first fluid from the first chamber; a second plunger for expelling the second fluid from the second chamber; and a manually operable valve positioned in the lower barrel configured to be moved between a first position, where fluid flow from the second chamber through the valve to the fluid port for expelling the second fluid from the syringe is permitted, and a second position, where fluid flow from the first chamber through the valve to the fluid port for expelling the first fluid from the syringe is permitted.

Clause 43: The syringe of clause 42, wherein the first chamber and the second chamber extend axially through the upper barrel, with a longitudinal axis of the first chamber being substantially parallel to a longitudinal axis of the second chamber.

Clause 44: The syringe of clause 42 or clause 43, wherein, with the manually operable valve in the first position, fluid flow from the first chamber through the valve is prevented, and with the valve in the second position, fluid flow from the second chamber through the valve is prevented.

Clause 45: The syringe of clause 44, further comprising at least one seal covering the open distal end of the upper barrel, the at least seal comprising a first opening for permitting the first fluid to flow from the first chamber to the valve, when the valve is in the second position, and a second opening for permitting the second fluid to flow from the second chamber through the valve, when the valve is in the first position.

Clause 46: The syringe of clause 45, wherein the at least seal comprises a proximal sheet positioned between the distal end of the upper barrel and the valve, and a distal sheet positioned between the valve and a distal end of the lower barrel.

Clause 47: The syringe of any of clauses 42-46, wherein the upper barrel is connected to the lower barrel by ultrasonic welding.

Clause 48: The syringe of any of clauses 42-47, wherein the first plunger comprises a first stopper in the first chamber and a first rod connected to and extending from the first stopper through the proximal end of the upper barrel.

Clause 49: The syringe of clause 48, wherein the first plunger rod comprises a tab extending from the first plunger rod that contacts a portion of the upper barrel as the first plunger rod is being moved from the first chamber, thereby providing feedback that an initial volume of the first fluid has been expelled from the first chamber.

Clause 50: The syringe of any of clauses 42-49, wherein the manually operable valve comprises a body comprising a through hole defining a flow channel which is fluidly connected to the second chamber when the valve is in the first position and fluidly connected to the first chamber when the valve is in the second position.

Clause 51: The syringe of clause 50, wherein the body comprises a rotating disc and a tab extending through an opening of the lower barrel for rotating the disc between the first position and the second position.

Clause 52: A syringe comprising: a barrel comprising an open proximal end, a distal end comprising a frustoconical portion and a fluid port, a cylindrical sidewall between the proximal end and the distal end, and a partition extending axially through the barrel separating the barrel into a first chamber configured to contain the first fluid and a second chamber configured to contain the second fluid; a first plunger for expelling the first fluid from the first chamber; a second plunger for expelling the second fluid from the second chamber; and a compressible bellows disposed in the first chamber configured to move between an unbiased position, where fluid flow from the first chamber is prevented, and a biased position, where fluid flow from the first chamber to the fluid port is permitted.

Clause 53: The syringe of clause 52, wherein the first chamber and the second chamber extend axially through the barrel with a longitudinal axis of the first chamber being substantially parallel to a longitudinal axis of the second chamber.

Clause 54: The syringe of clause 52 or clause 53, wherein the first plunger comprises a first stopper comprising a substantially flat distal surface in the first chamber and a first rod connected to and extending from the first stopper through the proximal end of the barrel.

Clause 55: The syringe of clause 54, wherein the second plunger comprises a second stopper comprising a half conical distal surface in the second chamber and a second rod connected to and extending from the second stopper through the proximal end of the barrel.

Clause 56: The syringe of clause 55, wherein the barrel comprises at least one slot for the flow of the first fluid from the first chamber to the fluid port, and wherein the at least one slot is positioned such that when the compressible bellows is in the unbiased position, fluid flow into the at least one slot is prevented and when the compressible bellows is in the biased position, fluid flow through the at least one slot to the fluid port is permitted.

Clause 57: The syringe of clause 56, wherein the at least one slot is an opening extending through the partition of the barrel, and wherein, when the second stopper is in a distal-most position in the second chamber, the second stopper seals a portion of the at least one slot preventing the first fluid from flowing into the second chamber.

Clause 58: The syringe of any of clauses 52-57, wherein the compressible bellows comprises a half conical distal surface configured to be positioned in the frustoconical portion of the barrel and at least one radially extending rib configured to contact an inner surface of the cylindrical sidewall of the barrel to seal the first fluid within the first chamber.

### BRIEF DESCRIPTION OF THE DRAWINGS

FIG. 1A is a perspective view of a multi-chamber syringe, according to an aspect of the present disclosure.

FIG. 1B is an exploded view of the syringe of FIG. 1A.

FIG. 2A is a perspective view of a lower portion of a stopper of the syringe of FIG. 1A, according to an aspect of the present disclosure.

FIG. 2B is a perspective view of a seal for the valve of the syringe of FIG. 1A, according to an aspect of the present disclosure.

FIG. 2C is a perspective view of a valve of the syringe of FIG. 1A, according to an aspect of the present disclosure.

FIG. 2D is a perspective view of a distal or lower portion of a barrel of the syringe of FIG. 1A, according to an aspect of the present disclosure.

FIG. 3A is a cross-sectional view of the syringe of FIG. 1A in an initial position.

FIGS. 3B-3E are cross-sectional views of the syringe of FIG. 1A in use.

FIG. 3F is a cross-sectional view of the syringe of FIG. 1A after expelling fluids from the syringe.

FIGS. 4A and 4B are cross-sectional views of portions of a sidewall of the syringe of FIG. 1A showing a welded joint between portions of the barrel, according to an aspect of the disclosure.

FIG. 5 is a flow chart showing a method of use for a multi-chamber syringe for sequential fluid expulsion, according to an aspect of the present disclosure.

FIG. 6A is a perspective view of another example of a multi-chamber syringe, according to an aspect of the present disclosure.

FIG. 6B is a perspective view of a distal or lower portion of a barrel of the syringe of FIG. 6A.

FIG. 6C is a perspective view of the distal or lower portion of the syringe of FIG. 6A showing forces exerted on a valve of the syringe by fluid in the first chamber of the syringe.

FIG. 6D is a cross-sectional view of a distal end of the syringe of FIG. 6A showing a valve of the syringe in a compressed position.

FIG. 7A is a cross-sectional view of the syringe of FIG. 6A in an initial position.

FIGS. 7B and 7C are cross-sectional views of the syringe of FIG. 6A in use to aspirate and expel fluids from the syringe.

FIG. 7D is a cross-sectional view of the syringe of FIG. 6A after expelling the fluids from the syringe.

FIGS. 8A and 8B are perspective views of stoppers of the syringe of FIG. 6A, according to an aspect of the present disclosure.

FIGS. 9A and 9B are views of a valve of the syringe of FIG. 6A, according to an aspect of the disclosure.

FIGS. 10A and 10B are cross sectional views of a proximal portion of the syringe of FIG. 6A showing locking tabs extending between plunger rods of the syringe, according to an aspect of the present disclosure.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. As used herein, the term “proximal” refers to a portion or end of a device, such as a syringe or catheter, which is grasped, manipulated, or used by a practitioner or another user. The term “distal” refers to an end or portion of the device that is farthest away from the portion of the device that is grasped, manipulated, or used by the practitioner. For example, the “proximal end” of a catheter or IV line refers to the end including a fluid port that is connected to a fluid container, such as an IV bag or syringe. The “distal end” of the catheter or IV line refers to the end that is connected to the patient. However, it is to be understood that the invention may assume alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

With reference to the figures, the present disclosure is directed to multi-chamber syringes 10, 210 that expel a first fluid F1 and a second fluid F2 from a container or barrel through a fluid port or nozzle of the syringe 10, 210 to, for example, a vascular access device (VAD). The first fluid F1 can be a flush solution, such as saline and/or a heparin lock flush solution. An example of a saline flush solution is 0.9% sodium chloride USP for injection. An example of a heparin lock flush solution is 0.9% sodium chloride with 100 USP units of heparin sodium per mL or 10 USP units of heparin sodium per mL. Other flush solutions, as are known in the art, may also be used with the syringes 10, 210 of the present disclosure. The second fluid F2 can be a medical fluid, which, as used herein, can refer to a medication, a total parenteral nutrient (TPN) liquid, or another therapeutic agent used for treatment of chronic or acute conditions, as are known in the art. Exemplary therapeutic agents can include, for example, drugs, chemicals, biological or biochemical substances that, when delivered in a therapeutically effective amount to the patient, achieve a desired therapeutic effect.

In some examples, the syringes 10, 210 are configured for performing two steps or aspects of an injection procedure, specifically injecting the medical fluid following by the flush solution. In other examples, the syringes 10, 210 can be used for performing three steps or aspects of an injection procedure, specifically injection of a first volume of the flush solution (first fluid F1) to prime and/or confirm patency of a patient line (referred to herein as a primary or pre-flush), followed by injection of a volume of the medical fluid (second fluid F2), followed by injection of a second volume of the flush solution (the first fluid F1) to clean the patient line and to confirm that all of the medical fluid passes from the patient line to the patient (referred to herein as a post flush). The syringes 10, 210 of the present disclosure can also be used for sequential delivery of a first medication or therapeutic agent followed by a different second medication or therapeutic agent.

The syringes 10, 210 of the present disclosure allow a practitioner, such as a medical technician, nurse, physician assistant, physician, or other trained or untrained clinicians or medical caregivers, to administer, for example, a medication followed by a flush solution without needing to change syringes between delivery of the first fluid and the second fluid. Accordingly, pre-flush, medication injection, and post-flush can be performed immediately after one another avoiding delays caused by switching between different syringes or injection devices.

In some examples, the syringes 10, 210 are provided as a partially pre-filled syringe, where a chamber of the syringe 10, 210 is filled with the flush solution during manufacturing. The partially pre-filled syringe 10, 210 can include caps, clips, retainers, and/or other packaging to hold the plunger rod in place and to ensure that the flush solution does not leak from the partially pre-filled syringe 10, 210 at unexpected times, such as during transport.

The syringes 10, 210 of the present disclosure are also configured to allow the practitioner to aspirate a medical fluid into the syringe 10, 210 prior to fluid delivery to the patient. For example, the practitioner can insert a nozzle or needle of the syringe 10, 210 into a vial containing the medical fluid and then aspirate the medical fluid into a fluid chamber of the syringe 10, 210 by moving the plunger rod of the syringe 10, 210 in a proximal direction. After the medical fluid is aspirated into the fluid chamber of the syringe 10, 210, delivery of the first fluid F1 followed by the second fluid F2 can occur by connecting the syringe 10, 210 to a fluid port of a VAD and then expelling the first fluid and the second fluid in sequence.

By eliminating the need to clean or disinfect portions of the VAD between delivery of the first fluid and the second fluid, the syringes 10, 210 of the present disclosure simplify the fluid administration procedure, providing substantial time savings compared to conventional fluid administration practices. The syringes 10, 210 of the present disclosure also reduce infection risk and allow for flushing of the VAD immediately following administration of the medical fluid, which may prevent drug occlusion in the VAD. Further, the syringes 10, 210 of the present disclosure have low or zero dead space, meaning that all medical fluid contained in a first chamber of the syringe 10, 210 is flushed from the syringe 10, 210 as the second fluid (e.g., the flush solution) moves through the syringe 10, 210 to the VAD.

### Multi-Chamber Syringe for Sequential Drug Delivery

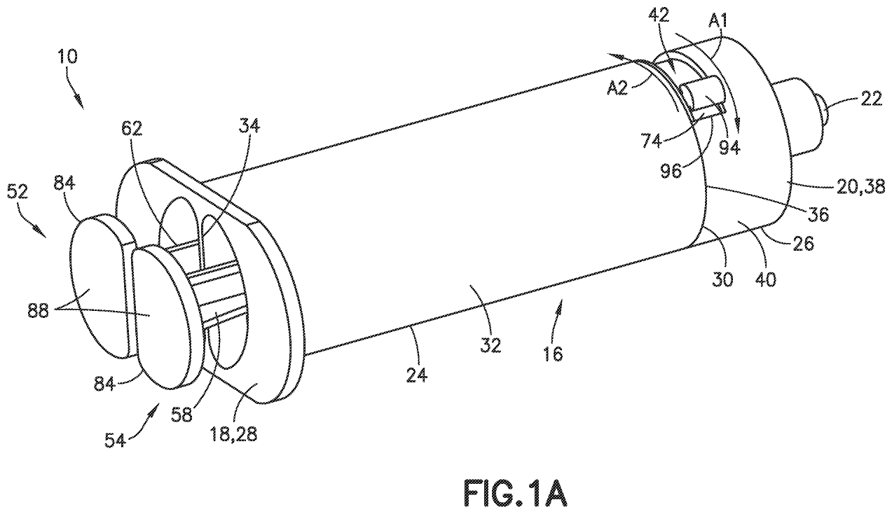

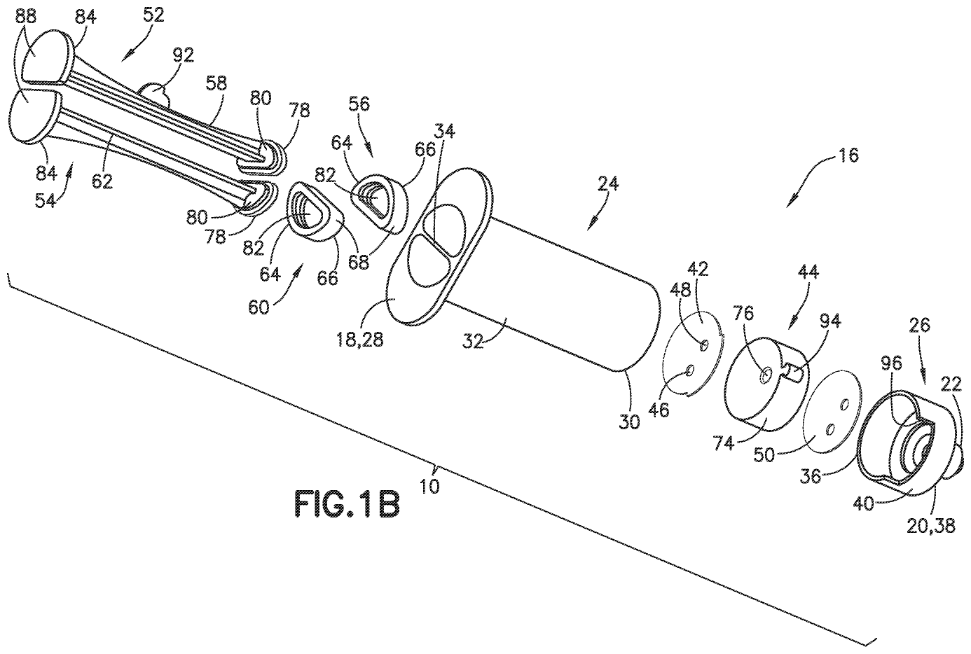

FIGS. 1A-4B illustrate an example of a multi-chamber syringe 10 for sequential expulsion of a first fluid F1 contained in a first fluid chamber 12 and a second fluid F2 contained in a second fluid chamber 14. For example, the syringe 10 can be configured to perform a two-step fluid delivery comprising expelling a predetermined volume of the second fluid F2 from the second chamber 14 followed by expelling a predetermined volume of the first fluid F1 from the first chamber 12. In other examples, the syringe 10 can be configured for three-step fluid delivery comprising ejecting a predetermined volume of the first fluid F1 from the first chamber 12, followed by ejecting a predetermined volume of the second fluid F2 from the second chamber 14, followed by ejecting a remaining volume of the first fluid F1 from the first chamber 12.

As previously described, in some examples, the first fluid F1 can be a flush solution, such as saline solution and/or an anticoagulant, such as heparin. The type of flush solution and amount of flush solution contained in the first chamber 12 may vary depending, for example, on the specific type of catheter or IV line being used. In some examples, the syringe 10 is partially prefilled containing, for example, between about 1 mL and 20 mL of the first fluid F1 or, preferably, between about 5 mL and about 10 ml of the first fluid F1. The second fluid F2 can be a medical fluid, such as a drug or another therapeutic agent intended for delivery to a patient through a VAD, such as a catheter or IV line.

In some examples, the syringe 10 comprises a container 16 or housing, which defines or encloses the first chamber 12 and the second chamber 14. The container 16 can comprise an open proximal end 18 and a distal end 20 comprising a fluid port 22 or nozzle for expulsion of the fluids F1, F2 from the container 16. The fluid port 22 of the container 16 can be a connector, such as a luer connector, threaded connector, or snap connector, configured to be connected to a needle for accessing, for example, an interior of a medical vial containing a medical fluid. The fluid port 22 can also be configured to be connected directly or indirectly to a fluid port, valve, or another terminal access portion of a VAD. For example, a common type of fluid port of a VAD is a pierceable septum or pre-slit septum made of rubber or another elastomeric material, which permits insertion of a sharp or blunt needle cannula in order to infuse fluids or to withdraw fluids from a catheter of the VAD. Another common fluid port of a VAD is a valve, which does not require a needle for accessing the VAD. Instead, the valve can be activated by a frusto-conically shaped tip of the syringe barrel 16 to provide fluid communication between the interior of the container 16 and the VAD.

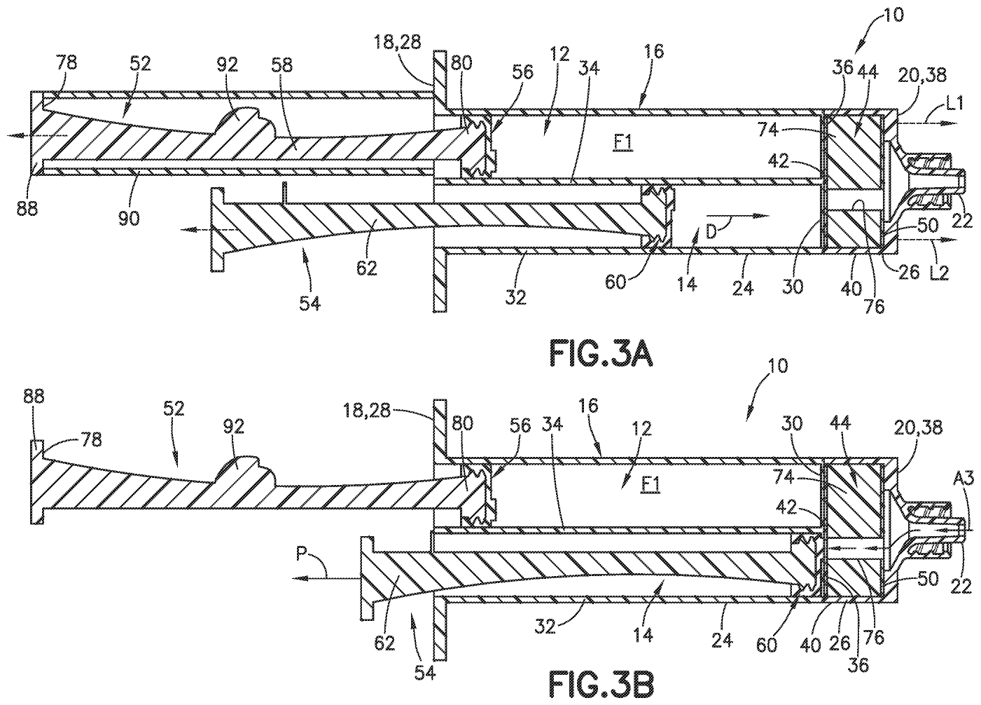

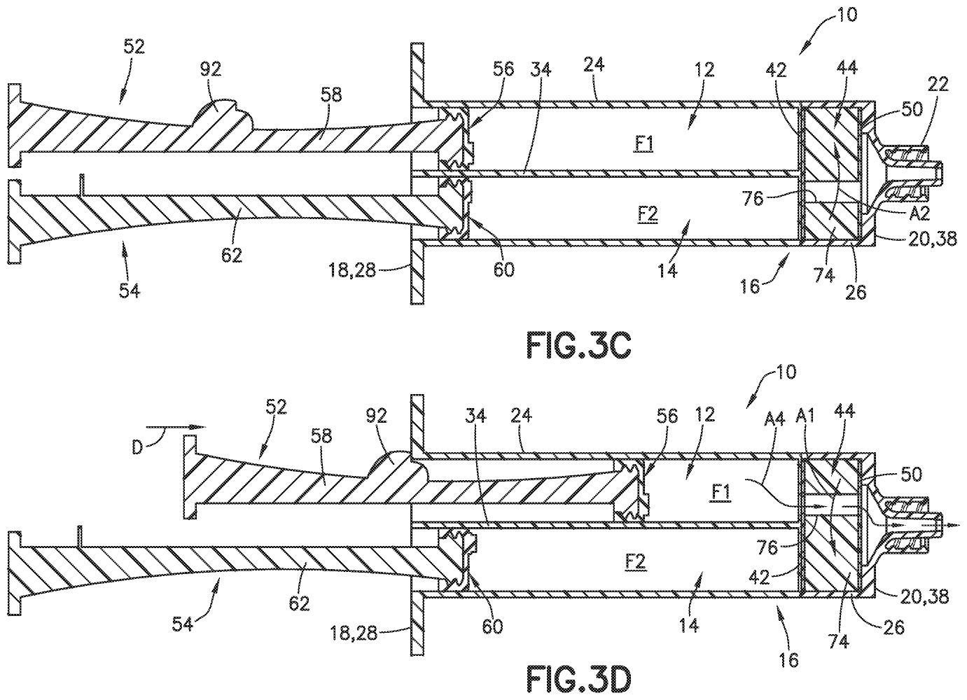

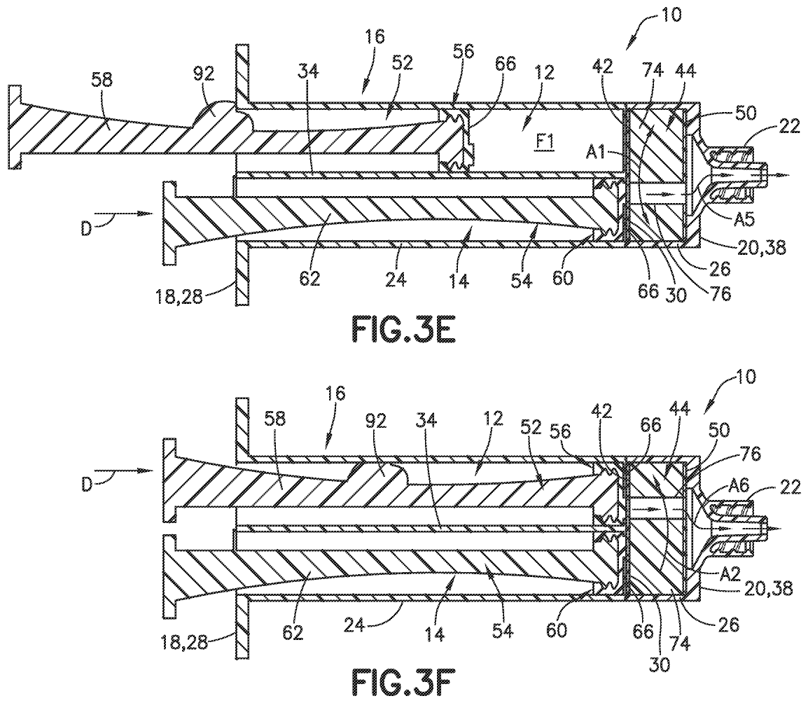

In some examples, the first chamber 12 and the second chamber 14 are parallel or substantially parallel cavities or spaces extending axially through the container 16. For example, a longitudinal axis L1 (shown in FIG. 3A) of the first chamber 12 can be parallel or substantially parallel to a longitudinal axis L2 (shown in FIG. 3A) of the second chamber 14. As used herein, “substantially parallel” means that the axis L1 is nearly parallel to the axis L2. For example, the axis L1 may be angled relative to the axis L2 by only a nominal amount, such as by less than 5 degrees. The chambers 12, 14 can be any convenient shape determined based on dimensions of the container 16 and/or other components of the syringe 10. For example, the first chamber 12 and the second chamber 14 can have a semi-circular cross-sectional shape, as well as an oval, circle, square, triangle, or rectangle shape.

In some examples, the container 16 is a syringe barrel, such as a barrel of a conventional fluid-delivery syringe used for medical procedures made by, for example, an injection molding process. For example, the container 16 or barrel can be substantially similar in shape, size, and configuration to barrels of syringes used for administering a flush solution to a VAD, as are known in the art. The container 16 or barrel can be a cylindrical or elliptical prism structure formed from a rigid thermoplastic material, such as polyester, polycarbonate, polypropylene, polyethylene, polyethylene terephthalate, acrylonitrile butadiene styrene, or other injection moldable or formable resin materials, as are known in the art. Exemplary barrels for flush syringes are described, for example, in U.S. Patent Appl. Pub. No. 2020/0061297, entitled “Flush Syringe Assembly with Controlled Pulsatile Flushing,” which is incorporated herein by reference in its entirety.

In other examples, the container 16 can be formed from multiple separately molded parts, which are joined together to form the container 16. For example, the container 16 can comprise an upper barrel 24 enclosing the first chamber 12 and the second chamber 14, and a lower barrel 26 connected to the upper barrel 24. As was the case with more conventional syringe barrels, the upper barrel 24 and the lower barrel 26 of the container 16 can be formed from at least one of polyester, polycarbonate, polypropylene, polyethylene, polyethylene terephthalate, or acrylonitrile butadiene styrene.

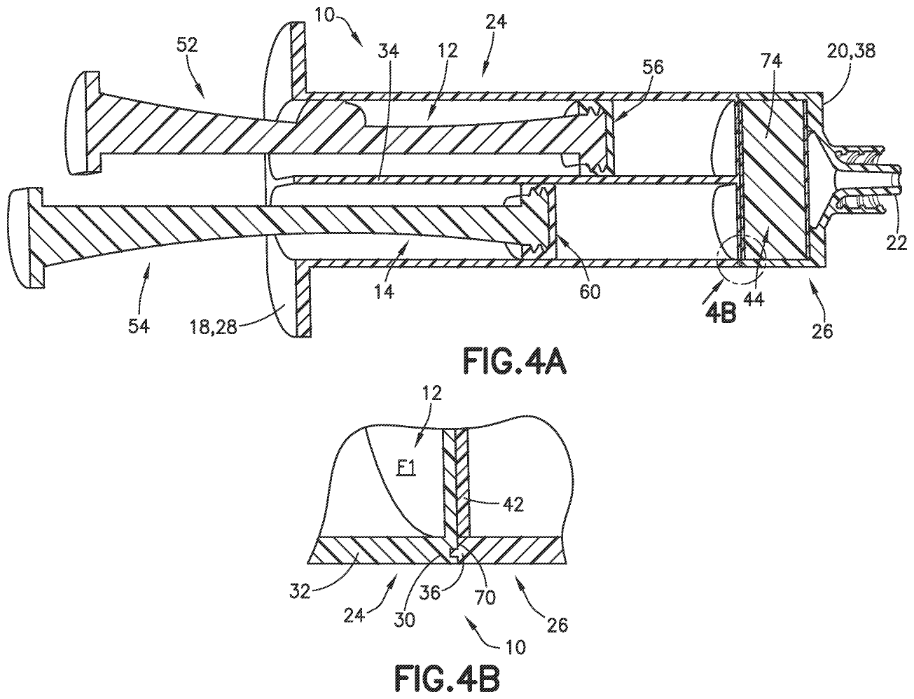

In some examples, the upper barrel 24 of the container 16 comprises an open proximal end 28, a distal end 30, and an annular sidewall 32 extending between the open proximal end 28 and the distal end 30. The distal end 30 of the upper barrel 24 can be open or can be substantially closed, but can include openings permitting fluid contained in the chambers 12, 14 to pass from the chambers 12, 14 through the distal end 30 of the upper barrel 24. The upper barrel 24 also includes an axially extending partition 34, such as a wall or baffle, extending from the open proximal end 28 to the distal end 30 of the upper barrel 24 separating the first chamber 12 from the second chamber 14. In some examples, the partition 34 is centrally positioned within the container 16 separating the container 16 into the first chamber 12 and the second chamber 14 having the same or substantially the same fluid volume. In other examples, the partition 34 can be off-centered or spaced apart from a longitudinal axis of the container 16 meaning that the chambers 12, 14 can contain different fluid volumes. The lower barrel 26 of the container 16 can comprise an open proximal end 36, a closed distal end 38 comprising the fluid port 22 of the container 16, and an annular sidewall 40 extending between the proximal end 36 and the distal end 38. The upper barrel 24 can be joined to the lower barrel 26 by any suitable fastener or adhesive, as are known in the art. In some examples, the upper barrel 24 and the lower barrel 26 are joined by ultrasonic welding. For example, as shown in FIGS. 4A and 4B, the proximal end 36 of the lower barrel 26 can be joined to the distal end 30 of the upper barrel 24 at an ultrasonic welded joint 70 (shown in FIG. 4B).

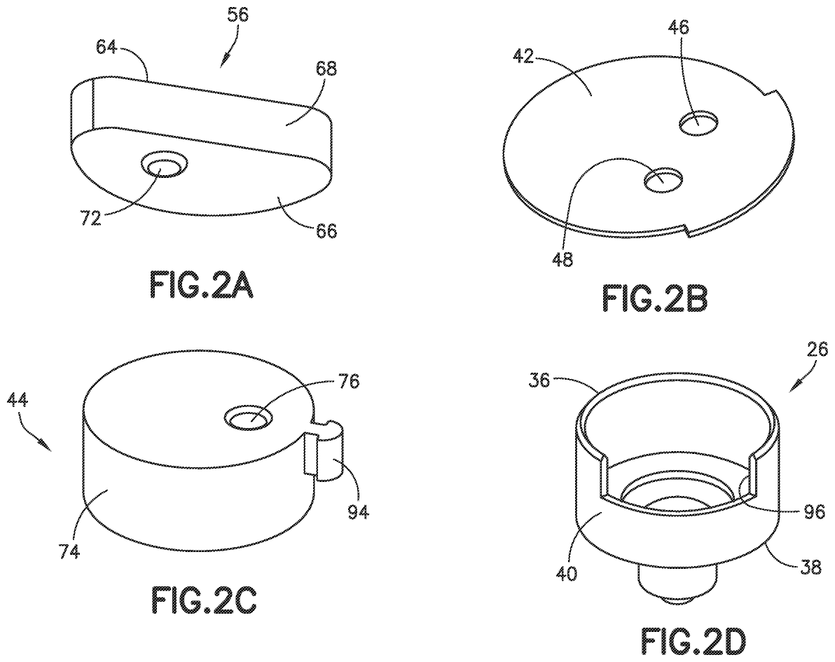

In some examples, the syringe 10 further comprises a seal, referred to herein as a proximal seal 42, covering the distal end 30 of the upper barrel 24 in order to seal the fluids within the chambers 12, 14 of the container 16. The proximal seal 42 can be a substantially flat sheet of a fluid-impermeable material, such as silicone, that is slightly smaller than the cross-section of the upper barrel 24. The proximal seal 42 can be positioned at the distal end 30 of the upper barrel 24 and held in place by a valve 44 positioned distal to the seal 42 or by contact between the seal 42 and, for example, an inner surface of the sidewall 40 of the lower barrel 26. In some examples, the proximal seal 42 includes a first opening 46 (shown most clearly in FIG. 2B) for permitting the first fluid F1 to flow from the first chamber 12 through the valve 44 to the fluid port 22. The proximal seal 42 can also include a second opening 48 (shown most clearly in FIG. 2B) for permitting the second fluid F2 to flow from the second chamber 14 through the valve 44. The syringe 10 can also include a distal seal 50, which is similar or identical in size and shape to the proximal seal 42, positioned between the valve 44 and the distal end 38 of the lower barrel 26.

The syringe 10 also includes multiple plungers configured to move through the chambers 12, 14 of the container 16 for aspirating the second fluid F2 into the second chamber 14 and/or for expelling the fluids F1, F2 from the chambers 12, 14 and through the fluid port 22 at the distal end 20 of the container 16. For example, the syringe 10 can include a first plunger 52 that moves through the first chamber 12 and a second plunger 54 that moves through the second chamber 14. The first plunger 52 can include a first stopper 56 (shown most clearly in FIG. 2A) in the first chamber 12 and a first plunger rod 58 connected to and extending from the first stopper 56 through the proximal end 18 of the container 16. The second plunger 54 can include a second stopper 60 in the second chamber 14 and a second plunger rod 62 connected to and extending from the second stopper 60 through the proximal end 18 of the container 16.

The first and second stoppers 56, 60 can have many features of conventional syringe stoppers or plungers, as are known in the art. For example, the stoppers 56, 60 can be formed from a thermoplastic elastomer material, such as polypropylene or polyethylene, as well as from synthetic or natural rubber (e.g., isoprene). The first stopper 56 and/or the second stopper 60 can include a proximal surface 64 or proximal end, a distal surface 66 or end, and an outer peripheral surface 68 extending between the proximal surface 64 and the distal surface 66. The outer peripheral surface 68 can be configured to seal against an inner surface of the sidewall 32 of the upper barrel 24 for moving fluids F1, F2 through chambers 12, 14 of the container 16. In some examples, the distal end or distal surface 66 of the stoppers 56, 60 can be substantially flat. Further, the distal surface 66 of the stoppers 56, 60 can be a semi-circular shape corresponding to the cross-sectional shape of the first chamber 12 and/or the second chamber 14. In some examples, as shown most clearly in FIG. 2A, the stoppers 56, 60 can also include a protrusion 72 configured to be inserted into and engage the openings 46, 48 of the proximal seal 42 in order to eliminate dead space in the openings 46, 48. For example, the first stopper 56 can include the protrusion 72 that inserts through the first opening 46 when the first stopper 56 is in a distal-most position in the first chamber 12 to ensure that all of the first fluid F1 is expelled from the first chamber 12 to the valve 44. In a similar manner, the second stopper 60 can include the protrusion 72 positioned to be inserted into the second opening 48 when the second stopper 60 is in the distal-most position to ensure that all of the second fluid F2 is expelled from the second chamber 14.

As previously described, the second stopper 60 can be configured to move through the second chamber 14 to aspirate fluid into the container 16. For example, movement of the second stopper 60 through the second chamber 14 in a proximal direction, as shown by arrow P in FIG. 3B, aspirates the second fluid F2 into the second chamber 14 through the fluid port 22 of the container 16. As described in further detail hereinafter, movement of the first stopper 56 through the first chamber 12 in a distal direction, as shown by arrow D in FIG. 3D, expels the first fluid F1 from the first chamber 12. In a similar manner, movement of the second stopper 60 distally through the second chamber 14 (as shown by arrow D in FIG. 3E) expels the second fluid F2, such as a medical fluid, from the second chamber 14.

The plungers 52, 54 can also include plunger rods, such as a first plunger rod 58 connected to the first stopper 56 and a second plunger rod 62 connected to the second stopper 60. The plunger rods 58, 62 can be, for example, injection molded parts formed from a rigid thermoplastic material, such as polyester, polycarbonate, polypropylene, polyethylene, polyethylene terephthalate, or another thermoplastic material, as are known in the art. The plunger rods 58, 62 can include a distal end 78 engaged to one of the stoppers 56, 60. For example, the distal ends 78 of the plunger rods 58, 62 can include an annular connector 80 that is inserted into a corresponding cavity 82 (shown in FIG. 1B) or slot on the proximal surface 64 of the stoppers 56, 60. The plunger rods 58, 62 also include a proximal end 84 protruding proximally from the proximal end 18 of the container 16 and a body extending between the proximal end 84 and the distal end 78 of the plunger rods 58, 62. The proximal end 84 of the plunger rods 58, 62 can include a thumb press plate 88 for manipulating the plunger rod 58, 62 to move the stoppers 56, 60 through the container 16. The body of the plunger rods 58, 62 can have a variety of cross-sectional shapes and configurations within the scope of the present disclosure. For example, the body can have a generally cross shaped cross-section. In other examples, the cross-section of the plunger rods 58, 62 can be an I-beam shape, hollow circle, hollow square, hollow rectangle, or L-shaped. In some examples, the syringe 10 can also include a removable plunger cap 90 (shown in FIG. 3A) engaged between the thumb press plate 88 of one or both of the plunger rods 58, 62 and the proximal end 18 of the container 16 for preventing movement of the plunger rods 58, 62 until the removable plunger cap 90 is removed from the syringe 10. For example, as shown in FIG. 3A, the removable cap 90 can be engaged to the first plunger rod 58 for restricting movement of the first plunger rod 58 through the first chamber 12.

In some examples, the first plunger rod 58 can further include a tab 92 configured to contact a portion of the container 16 as the first plunger rod 58 is being moved through the first chamber 12. The tab 92 can be positioned on the plunger rod 58 to provide tactile feedback that an initial volume of the first fluid F1 has been expelled from the first chamber 12. As described in further detail herein, the tactile feedback provided by the tab 92 helps the practitioner to perform a three-step fluid expulsion procedure. In particular, the practitioner can push the first plunger rod 58 and the first stopper 56 through the first chamber 12 of the container 16 until the practitioner feels the tactile feedback from the tab 92 indicating that the initial volume of the first fluid F1, such as a volume of flush solution sufficient to prime or confirm patency of a patient line, is expelled from the container 16. The practitioner can then move the second plunger rod 62 and second stopper 60 through the second chamber 14 in order to expel the second fluid F2, such as the medical fluid, to the patient line. Finally, the practitioner can apply sufficient force to the first plunger rod 52 to cause the tab 92 to deform or bend, allowing the first plunger rod 52 and stopper 56 to move through the first chamber 12 to expel a remaining volume of the first fluid F1 from the first chamber 12.

The syringe 10 also includes the valve 44, which is positioned in the container 16, configured to control sequential fluid flow from the first chamber 12 and from the second chamber 14. The valve 44 can be positioned in the lower barrel 26 of the container 16 between the proximal seal 42 and the distal seal 50. The valve 44 can be configured to permit fluid flow from either the first chamber 12 or the second chamber 14 through the valve 44 to the fluid port 22 of the container 16. More specifically, the valve 44 can be configured to transition between a first position (shown in FIGS. 3A-3C and FIG. 3E), where fluid flow from the first chamber 12 through the valve 44 is prevented, and a second position (shown in FIGS. 3D and 3F), where fluid flow from the first chamber 12 through the valve 44 to the fluid port 22 for expelling the first fluid F1 from the container 16 is permitted. Further, in some examples, with the valve 44 in the first position, fluid flow from the second chamber 14 through the valve 44 to the fluid port 22 for expelling the second fluid F2 from the container 16 is permitted. Similarly, with the valve 44 in the second position, fluid flow from the second chamber 14 through the valve 44 is prevented. Also, with the valve 44 in the first position, moving the second plunger 54 proximally through the second chamber 14 aspirates the second fluid F2 into the second chamber 14.

With continued reference to FIGS. 1A-3F, in some examples, the valve 44 can comprise a body 74 comprising a channel 76, which is fluidly connected to the second chamber 14, when the valve 44 is in the first position, and fluidly connected to the first chamber 12, when the valve 44 is in the second position. The body 74 can be a cylindrical structure, such as a rotating disc. In some examples, the body 74 or rotating disc is an injection molded part made from a rigid plastic. The body 74 or rotating disc can include a tab or knob 94 that is configured to be grasped by the practitioner for rotating the body 74 or disc to move the valve 44 between the first position and the second position. As shown in FIG. 1A, the tab or knob 94 can extend through an opening 96 in the sidewall 40 of the lower barrel 26. The practitioner can grasp or press against the tab or knob 94 to rotate the body 74 in a first direction (shown in arrow Al in FIG. 1A), which moves the body 74 towards the first position. Conversely, the practitioner can grasp or press against the tab or knob 94 to rotate the knob in a second direction (shown by arrow A2 in FIG. 1A), which moves the body 74 of the valve 44 to the second position. In some examples, the tab or knob 94 may be rotated by a predetermined angular distance, such as from 80 degrees to 120 degrees or, preferably an angular distance of about 100 degrees to move the body 74 or rotating disc between the first position and the second position.

As described in further detail herein, the syringe 10 may initially be provided in a partially filled state, as shown in FIG. 3A, with the first fluid F1, such as the flush solution, in the first chamber 12. In this initial or partially filled position, the first stopper 56 is positioned in the first chamber 12 near the proximal end 18 of the container 16, such that the first chamber 12 contains a volume of the first fluid F1 of about 1 mL to 20 mL, or preferably about 5 mL to 10 mL. The valve 44 is in the first position to prevent the first fluid F1 in the first chamber 12 from being expelled from the first chamber 12. The second stopper 60 can be inserted in the second chamber 14 in any position. When ready for use, the practitioner can move the second stopper 60 to a distal-most position seated against the distal end 30 of the upper barrel 24 with the protrusion 72 extending from the distal surface 66 of the second stopper 60 protruding at least partially through the first opening 46 of the proximal seal 42. The practitioner can then move the second stopper 60 in the proximal direction (shown by arrow P in FIG. 3B) to aspirate the second fluid F2, such as the medical fluid, into the second chamber 14, thereby fully filling the syringe 10, as shown in FIG. 3C. Once the syringe 10 is fully filled with both the first fluid F1 and the second fluid F2, the practitioner can use the plungers 52, 54 and valve 44 to sequentially control expulsion of the first fluid F1 and the second fluid F2 from the syringe 10, as described in detail hereinafter.

### Method of Expelling Fluid From a Multi-Chamber Syringe

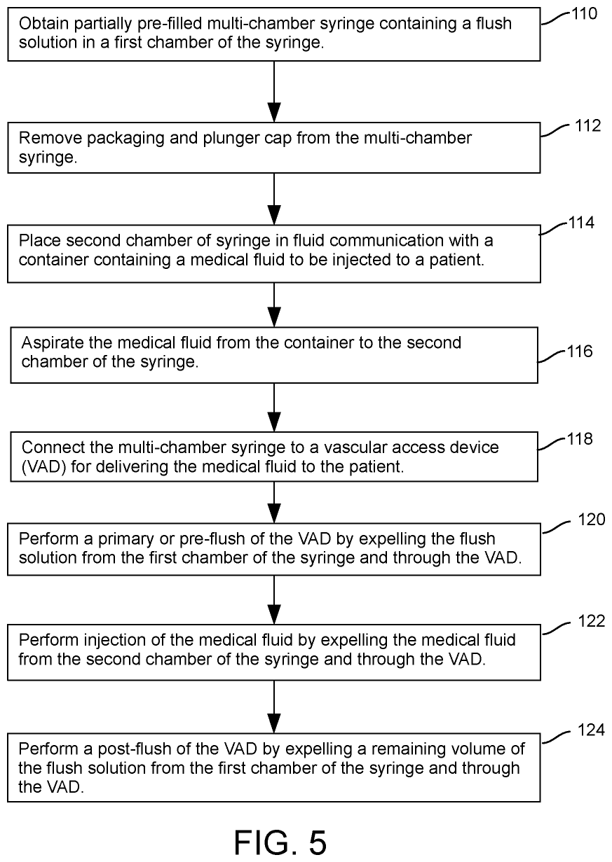

As previously described, the syringe 10 is used for sequential expulsion of fluids, such as expulsion of an initial volume of a flush solution, followed by expulsion of a volume of a medical fluid, followed by expulsion of a remaining volume of the flush solution from the syringe 10 to the VAD. A flow chart illustrating steps for a sequential fluid expulsion process using the syringe 10 is shown in FIG. 5.

As shown in FIG. 5, at step 110, a practitioner initially obtains a partially filled syringe 10, in which the first chamber 12 of the syringe 10 is filled with a predetermined volume of the first fluid F1, such as the flush solution. For example, the partially filled syringe 10 can contain about 1 mL to about 20 mL or, preferably, about 5 mL to 10 ml of the first fluid F1 or flush solution. The valve 44 is in the first position to prevent the flow of the first fluid F1 or flush solution from the first chamber 12. The syringe 10 in the initial or partially-filled position is shown in FIG. 3A. At step 112, the practitioner prepares the syringe 10 for use by, for example, removing any packaging from the syringe 10 and removing the plunger cap 90, if present, that holds the plunger rods 58, 62 in place. The practitioner may also move the second plunger rod 62 in the distal direction, shown by arrow D in FIG. 3A, to fully seat the second stopper 60 of the syringe 10 in its distal-most position. At step 114, the practitioner places the container 16 in fluid communication with an interior of a container containing the medical fluid to be delivered to the patient. For example, the practitioner may attach a needle (not shown) to the fluid port 22 at the distal end 20 of the container 16 and insert the needle into the medical container or vial containing the medical fluid to be injected to the patient.

At step 116, the practitioner aspirates the medical fluid into the second chamber 14 of the container 16. For example, the practitioner may grasp the second plunger rod 62 and move the second plunger rod 62 in the proximal direction, shown by arrow P in FIG. 3B, which moves the second stopper 60 in the proximal direction, thereby aspirating or drawing fluid into the second chamber 14, as shown by arrows A3 in FIG. 3B. The syringe 10 is shown in its fully filled position in FIG. 3C with the first fluid F1 in the first chamber 12 and the second fluid F2 in the second chamber 14.

Once a dose of the medical fluid to be delivered to the patient is drawn into the second chamber 14, at step 118, the practitioner removes the needle (not shown) from the fluid port 22 of the container 16 and connects the fluid port 22 to the VAD. For example, the practitioner may insert a nozzle of the container 16 into a corresponding port or valve of the VAD, thereby establishing fluid communication between the chambers 12, 14 of the container 16 and a lumen of the VAD.

At step 120, once the syringe 10 is appropriately connected to the VAD, the practitioner may perform a primary or pre-flush of the patient line. In order to perform the primary or pre-flush, the practitioner first moves the valve 44 to the second position by, for example, grasping the knob 94 (shown in FIGS. 1A, 1B, and 2C), which establishes fluid communication between the first chamber 12 and the fluid port 22 of the container 16. Specifically, as shown in FIG. 3C, the valve 44 can be rotated in a direction of arrow A2, such that the channel 76 moves from being in fluid communication with the second chamber 14 to being in fluid communication with the first chamber 12. The practitioner then grasps the first plunger rod 58 and pushes the first plunger rod 58 in the distal direction (shown by arrow D in FIG. 3D), which causes the first stopper 56 to move distally through the first chamber 12, forcing the first fluid F1 from the first chamber 12 as shown by arrows A4 in FIG. 3D. The practitioner continues to move the first stopper 56 distally through the first chamber 12 until the tab 92 of the first plunger rod 58 contacts the container 16, as shown in FIG. 3D. As previously described, the contact between the tab 92 and the container 16 provides tactile feedback to the practitioner indicating that the predetermined initial volume of the first fluid F1 has been expelled from the syringe 10 through the patient line.

One the primary or pre-flush has been performed, at step 122, the practitioner next expels the second fluid F2, which was aspirated into the second chamber 14 from the container 16 such as a medical vial, to the patient line. In order to expel the second fluid F2, the practitioner first moves the valve 44 to the first position, as shown by arrow Al in FIG. 3D, which establishes fluid communication between the second chamber 14 and the fluid port 22 of the container 16 through the valve 44. The practitioner then grasps the second plunger rod 62 and pushes the second plunger rod 62 in the distal direction (shown by arrow D in FIG. 3E), which causes the second stopper 60 to move distally through the second chamber 14. Distal movement of the second stopper 60 causes the second fluid F2 (e.g., the medical fluid) in the second chamber 14 to be expelled from the container 16, as shown by arrow A5 in FIG. 3E, to the VAD through the fluid port 22 or nozzle of the container 16. The practitioner continues to move the second plunger rod 62 in the distal direction until the second stopper 60 is seated against the proximal seal 42 at the distal end 30 of the upper barrel 24. The second stopper 60 is shown in this distal-most position in FIG. 3E.

At step 124, following expulsion of the second fluid F2 from the container 16, the practitioner next performs a second or post-flush to ensure that the full volume of the second fluid F2 has passed from the container 16 through the patient line and to clean and/or remove debris from the patent line. To perform the second or post-flush, the practitioner moves the valve 44 back to the second position, as shown by arrow A2 in FIGS. 3E and 3F, which establishes the fluid communication from the first chamber 12 to the fluid port 22 of the container 16 through the valve 44. The practitioner then grasps the first plunger rod 58 and pushes the first plunger rod 58 in the distal direction (shown by arrow D in FIG. 3F) with sufficient force to deform the tab 92, so that the first plunger rod 58 can be inserted farther into the first chamber 12. Once the tab 92 is depressed or deformed, the practitioner continues to push the first plunger rod 58 in the distal direction, which causes the first stopper 56 to move distally through the first chamber 12 expelling the first fluid F1 from the first chamber 12 through the fluid port 22, as shown by arrows A6 in FIG. 3F. The practitioner continues to move the first plunger rod 58 in the distal direction until the first stopper 56 is seated against the proximal seal 42 at the distal end 30 of the upper barrel 24, indicating that an entire remaining volume of the first fluid F1 or flush solution is expelled from the first chamber 12 and fluid port 22 to the patient line. The syringe 10 is shown in this final or end-of-use position in FIG. 3F, following expulsion of the full volume of the first fluid F1 and the full volume of the second fluid F2 from the container 16, with the distal surfaces 66 of the first stopper 56 and the second stopper 60 in contact with or nearly contacting the distal end 30 of the upper barrel 24 and/or the proximal seal 42.

### Multi-Chamber Syringe Including a Stopper With an Enclosed Fluid Passageway

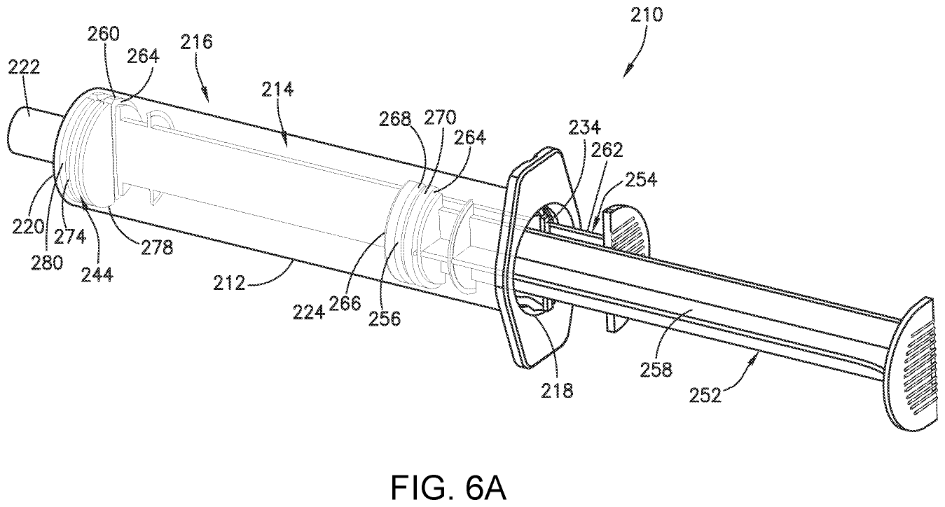

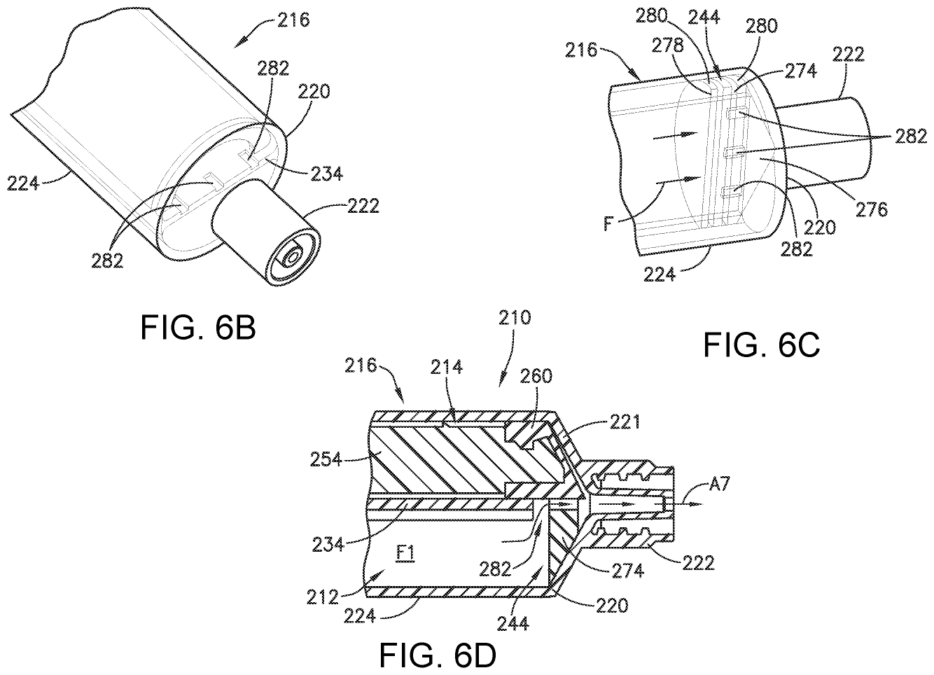

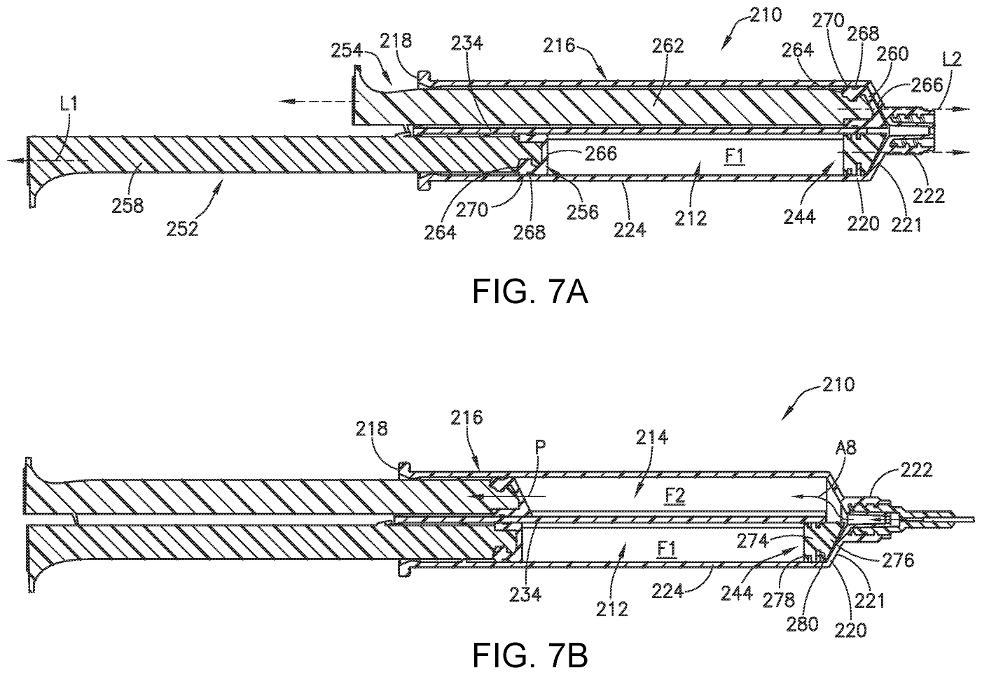

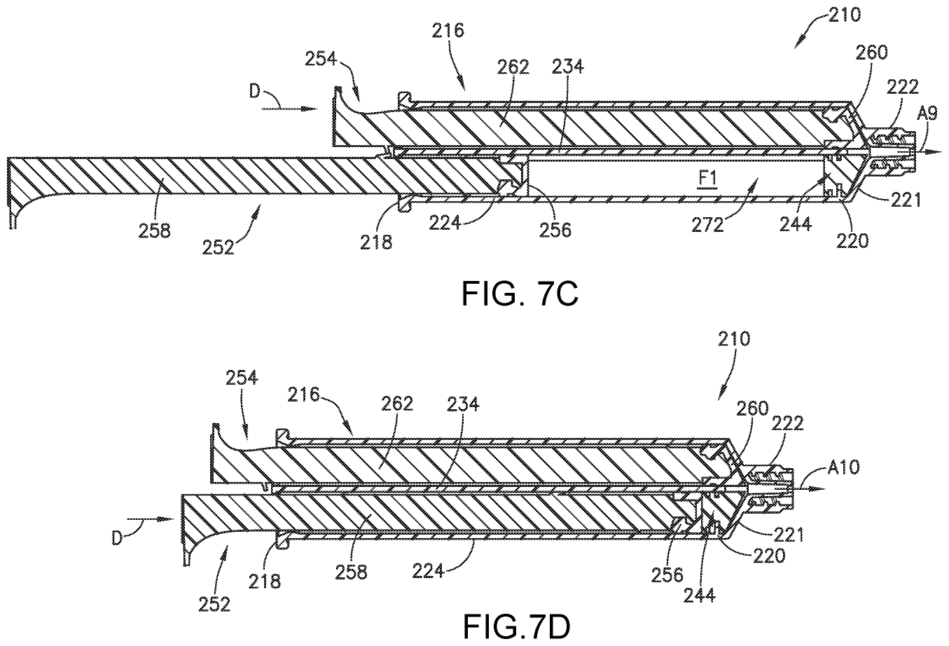

FIGS. 6A-7D illustrate another example of a multi-chamber syringe 210 for sequential delivery of a first fluid F1, such as a flush solution, and a second fluid F2, such as a medical fluid. The syringe 210 includes many of the components and features of the syringe 10 shown in FIGS. 1A-4B. In particular, the syringe 210 comprises a container that encloses or defines the first chamber 212 and the second chamber 214. The container can be a syringe barrel 216 having an open proximal end 218, a distal end 220 including a fluid port 222, and a sidewall 224 extending between the proximal end 218 and the distal end 220 of the barrel 216. The barrel 216 also includes a partition 234 or wall that separates an interior of the barrel 216 into the first chamber 212 and the second chamber 214. As shown, for example, in FIG. 7A, the first chamber 212 and the second chamber 214 are parallel or substantially parallel cavities or spaces extending axially through the barrel 216. For example, a longitudinal axis L1 (shown in FIG. 7A) of the first chamber 212 can be parallel or substantially parallel to a longitudinal axis L2 (shown in FIG. 7A) of the second chamber 214.

Unlike in previous examples, the syringe barrel 216 can be a single molded part similar in structure to a barrel of a conventional fluid delivery syringe. Also, the distal end 220 of the syringe barrel 216 can include a tapered portion 221 (shown in FIGS. 6D and 7A-7D) extending from the sidewall 224 of the barrel 216 to the fluid port 222 for guiding fluid from the chambers 212, 214 to the fluid port 222.

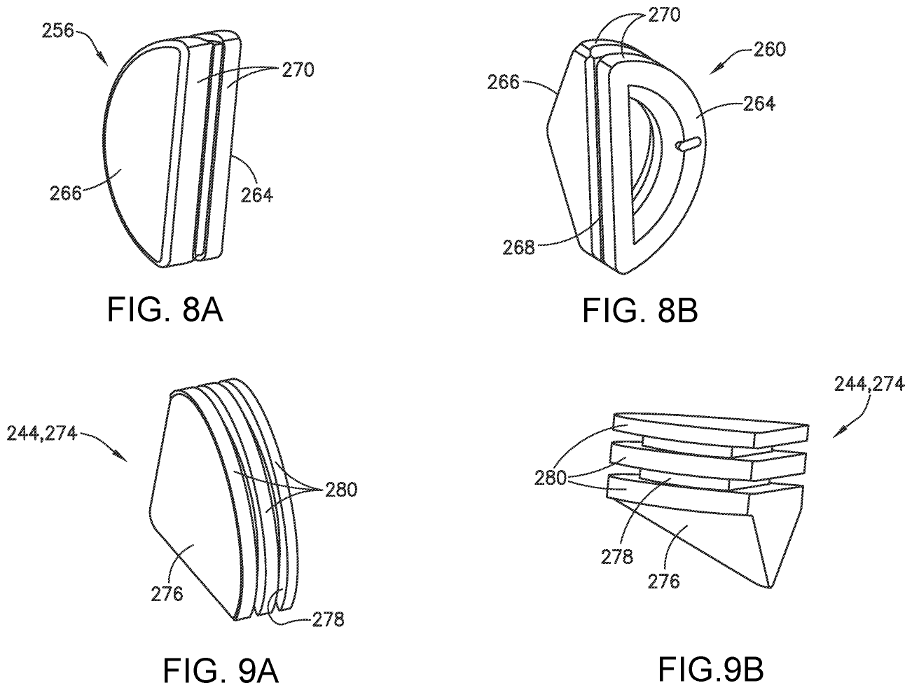

The syringe 210 further comprises the first plunger 252 for expelling the first fluid F1 from the first chamber 212 and the second plunger 254 for expelling the second fluid F2 from the second chamber 214. As in previous examples, the first plunger 252 comprises a first stopper 256, which is shown in FIG. 8A, and a first plunger rod 258. The second plunger 254 comprises a second stopper 260, which is shown in FIG. 8B, and a second plunger rod 262. The first stopper 256 comprises a proximal surface 264, a distal surface 266, and an annular outer peripheral surface 268 extending between the proximal surface 264 and the distal surface 266. The distal surface 266 of the first stopper 256 is flat. By contrast, the second stopper 260 includes a partially conical or frustoconical distal surface 266, which corresponds to a curvature or angle of the tapered portion 221 of the distal end 220 of the barrel 216. In particular, the partial or half-conical distal surface 266 of the second stopper 260 can be configured to push the second fluid F2 through the tapered portion 221 of the second chamber 214 ensuring that all or substantially all of the second fluid F2 is directed to the fluid port 222 and that none or only a nominal amount of the second fluid F2 remains in the second chamber 214 after use of the syringe 210 is completed.

As in previous examples, the syringe 210 also includes a valve 244 which controls sequential expulsion of the first fluid F1 and the second fluid F2 from the syringe 210. Unlike in previous examples, the valve 244 is an automatic valve that opens or closes due to forces (such as forces shown by arrow F in FIG. 6C) exerted on the valve 244 as the first plunger rod 258 and first stopper 256 move through the first chamber 212. Also, for the syringe 210, the valve 244 only controls fluid of the first fluid F1 from the first chamber 212. Specifically, when the valve 244 is in a first or closed position (shown in FIGS. 7A-7C), fluid flow from the first chamber 212 to the fluid port 222 of the barrel 216 through the valve 244 is prevented. When the valve 244 is in a second or open position (shown in FIGS. 6D and 7D), fluid flow from the first chamber 212 to the fluid port 222 through the valve 244 is permitted. For the syringe 210 of FIGS. 6A-7D, fluid flow from the second chamber 214 is not controlled by the valve 244 meaning that the second fluid F2 is permitted to flow from the second chamber 214 whenever the second plunger rod 262 moves the second stopper 260 through the second chamber 216 with sufficient force to move the fluid F2 through the fluid port 222 of the barrel 216.

As shown in FIGS. 6A-7D, as well as in FIGS. 9A and 9B, the valve 244 comprises a compressible body 274 or bellows which, in an unbiased or expanded position, prevents fluid flow from the first chamber 212 to the fluid port 222, and in a biased or compressed position, permits fluid flow from the first chamber 212 to the fluid port 222. Similar to the stoppers 256, 260, the compressible body 274 can be formed from a thermoplastic elastomer material, such as polypropylene or polyethylene, as well as from synthetic or natural rubber (e.g., isoprene). The compressible body 274 of the valve 244 includes a half or partially frusto-conical portion 276 that is positioned in the tapered or frustoconical portion 221 of the barrel 216. The compressible body 274 also includes a partially or semi-cylindrical portion 278 extending proximally from the frustoconical portion 276. The semi-cylindrical portion 278 can include one or more peripheral ribs 280 configured to contact and seal against an inner surface of the sidewall 224 of the barrel 216 to ensure that the first fluid F1 only passes through the valve 244 when the valve 244 is in the compressed or open position.

In some examples, the barrel 216 comprises structures, such as channels or grooves, for conducting the first fluid F1 through the valve 244 when the body 274 is compressed. For example, as shown most clearly in FIGS. 6B and 6C, the barrel 216 can include slots 282 defining a flow channel for the fluid flow from the first chamber 212 to the fluid port 222. The slots 282 can be positioned such that when the compressible body 274 is in the expanded position fluid flow through the slots 282 is prevented. When the compressible body 274 is in the compressed position, as shown in FIG. 6D, fluid flow through the slots 282 to the fluid port 222 is permitted. In some example, as shown in FIGS. 6B and 6C, the slots 282 are opening, cut-out portions, or through-holes extending through the partition 234 of the barrel 216. When the compressible body 274 of the valve 244 is in the expanded state, the compressible body 274 prevents the fluid F1 from passing into the slots 282 and/or from the first chamber 212 to the second chamber 214. The compressible body 274 also prevents any of the second fluid F2 from passing through the slots 282 into the first chamber 212 and/or mixing with the first fluid F1. When the second stopper 260 is in a distal-most position in the second chamber 214 (as shown, for example, in FIGS. 7A, 7C, and 7D), the second stopper 260 seals a portion of the slots 282 preventing the first fluid F1 from flowing into the second chamber 214. For example, the distal surface 266 of the second stopper 260, which is positioned in the frustoconical portion 221 of the barrel 216, can contact the partition 234 sealing the slots 282 to separate the first chamber 212 from the second chamber 214.

As in previous examples, the syringe 210 can be initially provided in a partially filled state (shown in FIG. 7A) with the first chamber 212 filled with the first fluid F1, such as the fluid solution, and the second chamber 214 can be empty. The second chamber 214 is filled by aspirating the second fluid F2 from a container, such as a medical vial, into the second chamber 214 through the fluid port 222 by moving the second plunger rod 260 in the proximal direction (shown by arrow P in FIG. 7B), which draws the second fluid F2 into the second chamber 214, as shown by arrow A8 (in FIG. 7B). Once the syringe 210 is fully filled with the first fluid F1 in the first chamber 212 and the second fluid F2 in the second chamber 214 (as shown in FIG. 7B), the second fluid F2 is expelled from the syringe 210 by moving the second plunger rod 262 in the distal direction (shown by arrow D in FIG. 7C), which expels the second fluid F2 from the second chamber 214, as shown by arrow A9 in FIG. 7C. The practitioner continues to move the second plunger rod 262 through the second chamber 214 until the second stopper 260 is seated against the distal end 220 of the barrel 216 to ensure that all or substantially all of the second fluid F2 is expelled from the second chamber 214. The second stopper 260 is shown in the distal-most position in FIG. 7C.

After expelling the second fluid F2 from the second chamber 214, the practitioner performs a post-flush by expelling the first fluid F1 from the first chamber 212. To expel the first fluid F1, the practitioner grasps the first plunger rod 258 and pushes the first plunger rod 258 in a distal direction (shown by arrow D in FIG. 7D), which causes the first stopper 256 to move through the first chamber 212. Movement of the first stopper 256 through the first chamber 212 increases the fluid pressure in the first chamber 212 causing the compressible body 274 of the valve 244 to compress. As previously described, the compressible body 274 is shown in the compressed position in FIG. 6D. As the body 274 compresses, fluid flows through channels or flow paths, such as the slots 282 in the partition 234 shown in FIGS. 6B-6D. The practitioner continues to push the first plunger rod 258 and first stopper 256 through the first chamber 212 until the distal surface 266 of the first stopper 256 contacts or nearly contacts the compressible body 274, as shown in FIG. 7D, thereby ensuring that all or substantially all of the first fluid F1 in the first chamber 212 flows through the slots 282 past the valve 244 and to the fluid port 222, where the first fluid F1 is expelled from the syringe 210. Accordingly, the syringe 210 provides for sequential expulsion of the second fluid F2 followed by the first fluid F1. The syringe 210 is shown in its final position with both the first fluid F1 and the second fluid F2 expelled from the syringe 210 in FIG. 7D.

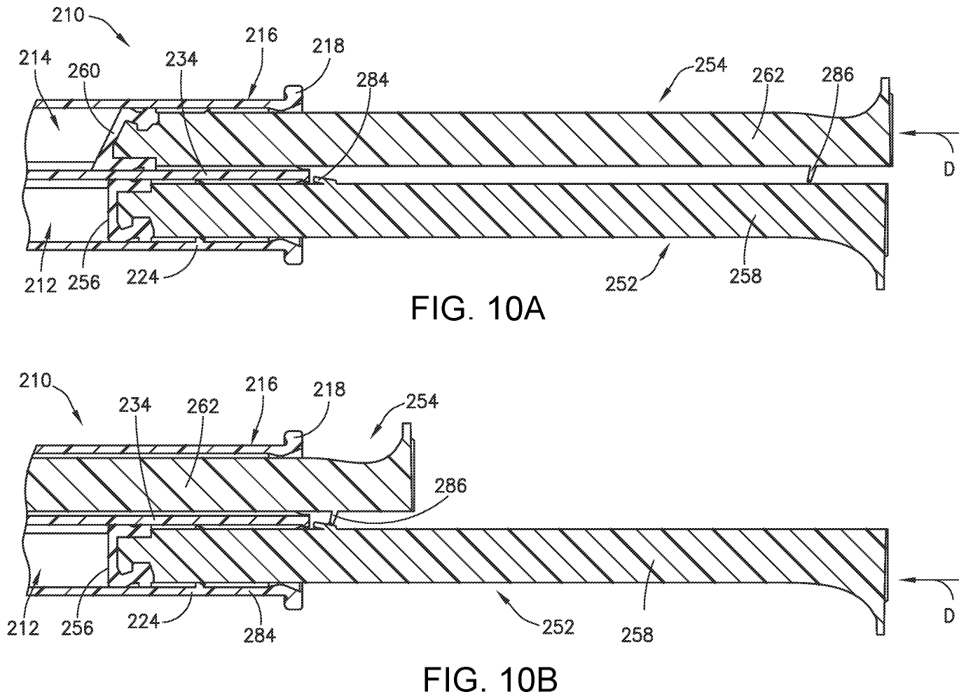

With reference to FIGS. 10A and 10B, in some examples, the plungers 252, 254 of the syringe 210 can include locking features for ensuring that the plunger rods 258, 262 are used in the correct order. For example, as shown in FIGS. 10A and 10B, the first plunger 252 can include a first tab 284 positioned proximate to a distal end and/or proximate to the stopper 256 of the first plunger rod 258. As used herein, “proximate to” the distal end and/or “proximate to” the stopper 256 can mean that the first tab 284 extends from a portion of the first plunger rod 258 that is closer to the distal end of the plunger rod 258 than to the proximal end of the plunger rod 258. As shown in FIG. 10A, the first tab 284 extends radially from the plunger rod 258 and is positioned to contact a portion of the barrel 216 to prevent the first plunger rod 258 from being moved distally through the first chamber 212 to expel the first fluid F1 from the first chamber 212 prior to expelling the second fluid F2 from the second chamber 214. Similarly, the second plunger 254 can include a second tab 286 extending from the second plunger rod 262. The second tab 286 can be positioned proximate to a proximal end of the second plunger rod 262. For example, the second tab 286 can be positioned on the second plunger rod 262 at a position closer to the proximal end of the plunger rod 262 than to the distal end of the plunger rod. The second tab 286 is configured to contact and depress the first tab 282 so that the first plunger rod 258 can be moved through the first chamber 212.

More specifically, in use, the plunger rods 258, 262 are in their proximal most positions in FIG. 10A, meaning that the syringe 210 is filled, with the first fluid F1 in the first chamber 212 and the second fluid F2 in the second chamber 214. In this positioned, as shown in FIG. 10A, the practitioner cannot insert the first plunger rod 258 farther into the first chamber 212 due to the contact between the first tab 284 and the partition 234 of the barrel 216. As previously described, the practitioner first moves the second plunger rod 262 through the second chamber 214 in the distal direction (shown by arrow D in FIG. 10A). Moving the second plunger rod 262 through the second chamber 214 in the distal direction (shown by arrow D) brings the second tab 286 into contact with the first tab 284. As shown in FIG. 10B, the contact between the first tab 284 and the second tab 286 depresses the first tab 284. Once the first tab 284 is depressed, the practitioner can move the first plunger rod 258 through the first chamber 212 in the distal direction (shown by arrow D in FIG. 10B), which causes the first fluid F1 to be expelled from the first chamber 212 as previously described.

While examples of the multi-chamber syringes and methods of the present disclosure are shown in the accompanying figures and described hereinabove in detail, other examples will be apparent to, and readily made by, those skilled in the art without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are to be embraced within their scope.

The invention claimed is

1. A multi-chamber syringe for sequential expulsion of at least a first fluid followed by a second fluid, the syringe comprising:
a container comprising a proximal end and a distal end comprising a fluid port for expulsion of the first fluid and the second fluid from the container, wherein the container defines a first chamber for containing the first fluid and a second chamber for containing the second fluid;
a first plunger for expelling the first fluid from the first chamber;
a second plunger for expelling the second fluid from the second chamber; and
at least one valve positioned in the container configured to transition between a first position, where fluid flow from the first chamber through the at least one valve is prevented, and a second position, where fluid flow from the first chamber through the at least one valve to the fluid port for expelling the first fluid from the container is permitted.

2. The syringe of claim 1, wherein the first chamber and the second chamber extend axially through the container, with a longitudinal axis of the first chamber being substantially parallel to a longitudinal axis of the second chamber.

3. The syringe of claim 1, wherein, with the at least one valve in the first position, fluid flow from the second chamber through the at least one valve to the fluid port for expelling the second fluid from the container is permitted, and with the at least one valve in the second position, fluid flow from the second chamber through the at least one valve is prevented.

4. The syringe of claim 1, wherein the container comprises an upper barrel comprising the first chamber and the second chamber, and a lower barrel connected to a distal end of the upper barrel enclosing the at least one valve.

5. The syringe of claim 4, wherein the upper barrel comprises: an open proximal end, a distal end, an annular sidewall extending between the open proximal end and the open distal end, and an axially extending partition extending from the open proximal end to the distal end of the upper barrel separating the first chamber from the second chamber.

6. The syringe of claim 5, further comprising at least one seal covering the open distal end of the upper barrel, the at least one seal comprising a first opening for permitting the first fluid to flow from the first chamber to the at least one valve, when the at least one valve is in the second position, and a second opening for permitting the second fluid to flow from the second chamber through the at least one valve, when the at least one valve is in the first position.

7. The syringe of claim 6, wherein the first plunger and/or the second plunger comprise a stopper having a flat distal surface and a protrusion extending from the flat distal surface positioned to insert into the first opening or the second opening to fully expel the first fluid from the first chamber or the second fluid from the second chamber.

8. The syringe of claim 1, wherein the first plunger comprises a first stopper in the first chamber and a first plunger rod connected to and extending from the first stopper through the proximal end of the container.

9. The syringe of claim 8, wherein the first plunger rod comprises a tab configured to contact a portion of the container as the first rod is being moved through the first chamber, thereby providing feedback that an initial volume of the first fluid has been expelled from the first chamber.

10. The syringe of claim 1, wherein the at least one valve comprises a body comprising a channel which is fluidly connected to the second chamber when the at least one valve is in the first position and fluidly connected to the first chamber when the at least one valve is in the second position.

11. The syringe of claim 10, wherein the body comprises a rotating disc and a knob extending through an opening of the container for rotating the disc between the first position and the second position.

12. The syringe of claim 1, wherein the container comprises a barrel comprising an annular sidewall, a frustoconical portion extending from the annular sidewall to the fluid port, and a partition extending through the barrel that separates the first chamber from the second chamber.

13. The syringe of claim 12, wherein the first plunger comprises a first stopper comprising a substantially flat distal surface and a first plunger rod connected to and extending proximally from the first stopper, and
wherein the second plunger comprises a second stopper comprising a half conical distal surface sized to be received within the frustoconical portion of the barrel and a second plunger rod connected to and extending proximally from the second stopper.

14. The syringe of claim 13, wherein the first plunger rod comprises a first tab positioned proximate to a distal end of the first plunger rod configured to contact the barrel to prevent the first plunger rod from being moved distally through the first chamber to expel the first fluid from the first chamber, and
wherein the second plunger rod comprises a second tab positioned proximate to a proximal end of the second plunger rod configured to contact and depress the first tab so that the first plunger rod can be moved through the first chamber.

15. The syringe of claim 12, wherein the at least one valve comprises a compressible body which, in an expanded position, prevents fluid flow from the first chamber to the fluid port, and in a compressed position permits fluid flow from the first chamber to the fluid port.

16. The syringe of claim 15, wherein the barrel comprises at least one slot defining a flow channel for the fluid flow from the first chamber to the fluid port, and wherein the at least one slot is positioned such that when the compressible body is in the expanded position, fluid flow through the at least one slot is prevented, and when the compressible body is in the compressed position, fluid flow through the at least one slot to the fluid port is permitted.

17. A pre-filled flushing syringe, comprising:
a container comprising a proximal end and a distal end comprising a fluid port for expulsion of the first fluid and the second fluid from the container, wherein the container defines a first chamber for containing the first fluid and a second chamber for containing the second fluid;
a first plunger for expelling the first fluid from the first chamber;
a second plunger for expelling the second fluid from the second chamber;
at least one valve positioned in the container configured to transition between a first position, where fluid flow from the first chamber through the at least one valve is prevented, and a second position, where fluid flow from the first chamber through the at least one valve to the fluid port for expelling the first fluid from the container is permitted; and
a predetermined volume of the first fluid disposed in the first chamber of the pre-filled syringe,
wherein the at least one valve is initially in the first position, thereby preventing the first fluid disposed in the first chamber from passing from the first chamber through the at least one valve.

18. The pre-filled syringe of claim 17, wherein the first fluid is a saline flush solution.

19. A syringe comprising:
a housing comprising an (i) upper barrel comprising an open proximal end, a distal end, a sidewall extending between the proximal end and the distal end, and a partition extending axially through the barrel separating the barrel into a first chamber configured to contain a first fluid and a second chamber configured to contain a second fluid, and (ii) a lower barrel connected to the distal end of the upper barrel comprising a fluid port;
a first plunger for expelling the first fluid from the first chamber;
a second plunger for expelling the second fluid from the second chamber; and
a manually operable valve positioned in the lower barrel configured to be moved between a first position, where fluid flow from the second chamber through the valve to the fluid port for expelling the second fluid from the syringe is permitted, and a second position, where fluid flow from the first chamber through the valve to the fluid port for expelling the first fluid from the syringe is permitted.

20. The syringe of claim 19, wherein the first chamber and the second chamber extend axially through the upper barrel, with a longitudinal axis of the first chamber being substantially parallel to a longitudinal axis of the second chamber.
